(19) United States
(12) Patent Application Publication
(10) Pub. No.: US 2016/0346218 A1
(43) Pub. Date: Dec. 1, 2016

(54) HYBRID HOLLOW MICROCAPSULE, SCAFFOLD FOR SOFT TISSUE INCLUDING SAME, AND METHODS OF PREPARING SAME

(71) Applicant: GWANGJU INSTITUTE OF SCIENCE AND TECHNOLOGY, Gwangju (KR)

(72) Inventors: Gi-Yoong TAE, Gwangju (KR); Raja RAJAMANICKAM, Gwangju (KR); Jong-Chul KIM, Gwangju (KR)

(21) Appl. No.: 15/166,189

(22) Filed: May 26, 2016

(30) Foreign Application Priority Data
May 27, 2015 (KR) 10-2015-0073755

(51) Int. Cl. A61K 9/48; A61L 27/22; B05D 3/00; A61L 27/18; A61L 27/20; A61L 27/12; A61L 27/02

(52) U.S. Cl. CPC A61K 9/4891; A61K 9/485; A61K 9/4866; A61K 9/4808; A61L 27/12; A61L 27/222; A61L 27/025; A61L 27/18; A61L 27/20; B05D 3/007; A61L 2430/34

(57) ABSTRACT

Disclosed is a method of preparing a hollow microcapsule using freezing of macroporous materials including a crosslinked inorganic particle network capable of elastically recovering from a highly compressed deformation state, and use of the same as a scaffold for soft tissue engineering and as a drug delivery system.

### BACKGROUND

1. Technical Field

The present invention relates to a hybrid hollow microcapsule, a scaffold for soft tissue including the same, and a method of preparing the same.

2. Description of the Related Art

In tissue engineering, macroporous biocompatible materials are used as a template for cellular growth and transplantation into an animal model in order to obtain desired biomedical effects. In order for the macroporous biocompatible materials to be used for tissue engineering, it is very important for the macroporous biocompatible materials to have mechanical properties similar to those of host tissues. Further, it was found that mechanical stimulus from the macroporous biocompatible materials might regulate stem cell differentiation.

Tissue engineering of soft tissues such as adipose tissues requires soft, elastic and resilient scaffolds like host tissues. For example, adipose tissues have a modulus of elasticity ranging from 3 kPa to 4 kPa. Scaffolds should maintain their internal structure when external forces are applied after implantation. Prior soft tissue engineering studies were mainly carried out using polymeric crosslinked macroporous scaffolds. Such polymeric scaffolds are soft, but do not have elastic resilience under high compressive strain. Furthermore, mechanical strength of these polymeric scaffolds is capable of being controlled simply through adjustment of crosslinking density of polymer chains.

In order to manufacture rigid scaffolds capable of being employed in bone regeneration, materials consisting of pure inorganic components are effective. Many studies using a porous hydroxyapatite as a scaffold for bone regeneration have been reported. These scaffolds are fragile and are not recovered once deformed. Further, these scaffolds are considered to have a slow rate of decomposition.

By a method of preparing a biomimetic hydroxyapatite/polymer complex, a fragile and porous material that is capable of being used as a bone substitute is obtained. The inventors of the present invention have reported that they could manufacture an elastic scaffold that has a network of inorganic particles onto which polyethyleneimine (PEI) is coated by a freezing method, is crosslinked by a diepoxy polyethylene glycol (PEG) crosslinking agent, and has amounts of inorganic materials of 85% or less. When these elastic scaffolds are used as scaffolds for tissue engineering, cytotoxicity due to released crosslinking agents can be problematic. Accordingly, there is a need for a method capable of manufacturing an elastic scaffold that does not include a crosslinking agent, is soft and has high amounts of inorganic materials.

There has been reported a method of synthesizing a polymer electrolyte hollow capsule by layer-by-layer adsorption of polymer electrolyte layers having opposite charges on a sacrificial core such as calcium carbonate micro-particles, silica particles, melamine resins, and the like. Mechanical properties of these polymeric polyelectrolyte multilayer (PEM) hollow capsules depend upon the number of PEM layers and the crosslinking density of polymer chains. Research into production of inorganic/organic hybrid hollow spheres having PEM shells on surfaces of inorganic nanoparticles has also been reported. Dmitry G. Shchukin et al., produced poly(allylamine hydrochloride) (PAH)/poly(sodium 4-sytrenesulfonate) (PSS) PEM capsules using Y₂O₃—FeO₃ and calcium phosphate, and Matthieu F. Bedard et al., reported production of polydiallyldimethylammonium chloride (PDADMAC)/PSS capsule shells including gold nanoparticles.

Mechanical properties of hollow capsules consisting of PEM were measured through measurement of force and deformation in the presence of mainly an atomic force microscope (AFM) colloidal probe, measurement of deformation due to osmotic pressure, and measurement of deformation of capsules occurring when pressed through a narrow channel. As a result of measurement of mechanical properties, PEM hollow capsules were found to have a recovery rate of up to 20% from deformation. In order to perform drug delivery by means of mechanical stimulation, the hollow capsules are required to have a recovery rate of up to 90% from compressive deformation.

### PRIOR ART DOCUMENTS

Patent Document 1: U.S. Pat. No. 8,623,085
Non-Patent Document 1: Langer R, Vacanti J P “Tissue engineering” Science 260 (5110): 920-926
Non-Patent Document 2: D. W. Hutmacher “Scaffolds in tissue engineering bone and cartilage” Biomaterials, 21 (24) (2000), pp. 2529-2543
Non-Patent Document 3: R. A. Marklein and J. A. Burdick, “Controlling Stem Cell Fate with Material Design” Adv. Mater., 2010, 22, 175-189.
Non-Patent Document 4: L. E. Flynn, “The use of decellularized adipose tissue to provide an inductive microenvironment for the adipogenic differentiation of human adipose-derived stem cells” Biomaterials, 2010, 31, 4715-4724.
Non-Patent Document 5: L. Flynn and K. A. Woodhouse, “Adipose tissue engineering with cells in engineered matrices” Organogenesis, 2008, 4, 228-235

### BRIEF SUMMARY

Various embodiments of the present invention provide a method of preparing a hollow microcapsule using freezing of macroporous materials including a crosslinked inorganic particle network capable of elastically recovering from a highly compressively deformed state, and use of the hollow microcapsule as a scaffold for soft tissue engineering and as a drug delivery system.

One aspect of the present invention relates to a hollow microcapsule, including: (a) a hollow core polymer layer, and (b) an organic-inorganic complex layer including inorganic nanoparticles and polymer for coating capsules on the surface of the hollow core polymer layer, wherein the organic-inorganic complex layer is a single organic-inorganic complex layer or a plurality of organic-inorganic complex layers formed in a layer-by-layer manner, and the core polymer layer and the polymer for coating capsules are crosslinked.

Another aspect of the present invention relates to a scaffold for soft tissue engineering including a hollow microcapsule according to various embodiments of the invention.

A further aspect of the present invention relates to a method of preparing a hollow microcapsule, including: (A) forming a core polymer layer on \textcircled{1} a positively charged sacrificial core or \textcircled{1} a negative charge-modified sacrificial core; (B) \textcircled{1} if the sacrificial core is the positively charged sacrificial core, alternately forming an inorganic nanoparticle layer and a polymer layer for coating capsules at least once on the core polymer layer, \textcircled{1} if the sacrificial core is the negative charge-modified sacrificial core, alternately forming an inorganic nanoparticle layer coated with a composition for coating inorganic nanoparticles and a polymer layer for coating capsules at least once on the core polymer layer; (C) crosslinking the core polymer and the polymer for coating capsules; and (D) removing the sacrificial core by etching.

According to various embodiments of the present invention, there are provided a method of preparing a hollow microcapsule using freezing of macroporous materials including a crosslinked inorganic particle network capable of elastically recovering from a highly compressively deformed state, and use of the hollow microcapsule as a scaffold for soft tissue engineering and as a drug delivery system. Elasticity of the microcapsule material is irrelevant to properties of particles used. Examples of the microcapsule materials may include microcapsule materials prepared by coating hydroxyapatite, silica nanoparticles and poly(lactic-co-glycolic acid) (PLGA) nanospheres as biocompatible inorganic nanoparticles with gelatin or chitosan as a natural biopolymer, wherein 1-ethyl-3-(3-dimethylaminopropyl)carbodiimide (EDC) and telechelic diepoxy or glutaraldehyde are employed as a crosslinking agent. Mechanical properties and decomposition properties of these materials can be controlled through control of crosslinking density. The recovery property of these scaffolds is very effective in loading cells into a scaffold. It was confirmed that these scaffolds are biocompatible through in vitro and in vivo experiments. Using the same method, it is possible to prepare an elastic hybrid hollow microcapsule through alternate adsorption of chitosan particles and 7 nm colloidal silica, hydroxyapatite or magnetite nanoparticles on calcium carbonate micro-particles capable of being etched with an ethylenediaminetetraacetic acid (EDTA) solution in a layer-by-layer (LbL) manner. The chitosan layer was crosslinked by glutaraldehyde or telechelic diepoxy, thereby stabilizing the microcapsule.

### BRIEF DESCRIPTION OF THE DRAWINGS

The above and other aspects, features, and advantages of the invention will become apparent from the detailed description of the following embodiments in conjunction with the accompanying drawings, in which:

FIG. 1A shows images of scaffolds comprising 10% hydroxyapatite (HAp), 1% gelatin and 4 mg 1-ethyl-3-(3-dimethylaminopropyl)carbodiimide (EDC) when swollen, compressed to ˜90%, and recovered;

FIG. 1B shows images of scaffolds comprising 10% hydroxyapatite (HAp), 1% gelatin and 0.1 mg EDC in the presence or absence of water;

FIG. 2A shows an image of a scaffold comprising 10% hydroxyapatite (HAp), 1% gelatin and 0.1 mg EDC;

FIG. 2B shows an image of a scaffold comprising 10% hydroxyapatite (HAp), 1% gelatin and 0.5 mg EDC;

FIG. 2C shows an image of a scaffold comprising 10% hydroxyapatite (HAp), 1% gelatin and 2 mg EDC;

FIG. 2D shows an image of a scaffold comprising 10% hydroxyapatite (HAp), 1% gelatin and 4 mg EDC;

FIG. 2E shows an image of a scaffold comprising 20% hydroxyapatite (HAp), 1% gelatin and 4 mg EDC;

FIG. 2F shows an image of a scaffold comprising 10% 0.5 μm-SiO₂, 1% gelatin and 4 mg EDC;

FIG. 3 shows thermogravimetric analysis graphs of scaffolds comprising bare hydroxyapatite nanoparticles (HAp), citrate-capped hydroxyapatite nanoparticles (Cit-HAp), gelatin-coated Cit-HAp (Gel-Cit-HAp), and 10% HAp, 1% gelatin/4 mg EDC. The graphs are depicted from 120° C. in order to avoid weight loss due to moisture;

FIG. 4A shows frequency sweeps of scaffolds comprising 10% HAp and 1% gelatin and rheological measurement results of scaffolds having four different amounts of EDC (namely, 0.5, 1, 1.5 and 2 mg of EDC);

FIG. 4B shows a graph of shear modulus change according to increase in EDC amount;

FIG. 4C shows swelling rate of scaffolds when water is used as a solvent;

FIG. 5 shows in-vitro enzymatic decomposition profiles of scaffolds under various conditions (0% of weight loss indicates that a scaffold is completely degraded into particles);

FIG. 6 shows a SEM photograph of a scaffold comprising 10% HAP, 1% gelatin and 0.5 mg EDC after seeding with NIH 3T3 and incubating for three days;

FIG. 7A to FIG. 7D show histological analysis for HAp-gelatin scaffolds subcutaneously injected into a mouse for two weeks;

FIG. 7A is a cross-sectional image of a hematoxylin-eosin stained scaffold (S: scaffold, dark purple; M: muscle) wherein an inserted photograph indicated by a dotted square on the right side is an image of a scaffold in an in-vivo implant state;

FIG. 7B is a cross-sectional image of a scaffold stained with Sirius red against collagen (collagen: dark red);

FIG. 7C is an enlarged view of C-section on the boundary surface (immune cells: dark purple dots without pale purple boundaries);

FIG. 7D is an enlarged view of D-section (settled cells: pale purple area with purple dots; blood vessel: bundle of bright red dots surrounded by purple area);

FIG. 8A shows an embodiment of a process for preparing hybrid hollow capsules having different sizes;

FIG. 8B shows another embodiment of a process for preparing hybrid hollow capsules having different sizes;

FIG. 8C shows optical images of hybrid hollow capsules having different sizes;

FIG. 9A shows a fluorescent optical image of an elastic hybrid hollow capsule prepared in Example 6-1 before squeezing the capsule through a narrow patch clamp;

FIG. 9B shows a fluorescent optical image of an elastic hybrid hollow capsule prepared in Example 6-1 after squeezing the capsule through a narrow patch clamp; FIG. 9C shows optical images of osmotically induced rupture performed at different PSS 70K Da Mw concentrations against hybrid hollow capsules (HHC); and.

FIG. 10 shows cycles of applied external forces, amounts of drugs released from each cycle, and cumulative graphs including representative fluorescent images of the corresponding capsules at each cycle of external forces (scale bar: 10 μm), respectively, which demonstrate experimental results for drug loading to hollow microcapsules and drug release by external forces performed in accordance with Example 9.

### DETAILED DESCRIPTION

Hereinafter, various aspects and exemplary embodiments of the present invention will be described in greater detail.

One aspect of the present invention relates to a hollow microcapsule, including: (a) a hollow core polymer layer having a hollow core, and (b) an organic-inorganic complex layer including inorganic nanoparticles and a polymer for coating capsules on a surface of the hollow core polymer layer, wherein the organic-inorganic complex layer is a single organic-inorganic complex layer or a plurality of organic-inorganic complex layers formed in a layer-by-layer manner, and the core polymer layer and the polymer for coating capsules are crosslinked.

In the present invention, an outermost layer of the organic-inorganic complex layer is a polymer layer for coating capsules. The core polymer layer and the polymer for coating capsules are preferably crosslinked in order to prevent loss of inorganic nanoparticles during washing.

The above aspect of the present invention may be accomplished by two exemplary embodiments as below.

According to a first exemplary embodiment of the present invention, the organic-inorganic complex layer may be composed of one or a plurality of organic-inorganic complex layers formed by alternately stacking (b1) an inorganic nanoparticle layer comprising inorganic nanoparticles and (b2) a polymer layer for coating capsules comprising the polymer for coating capsules at least once on the surface of the hollow core polymer layer.

According to a second exemplary embodiment of the present invention, the organic-inorganic complex layer may be composed of one or a plurality of organic-inorganic complex layers formed by alternately stacking (b1′) an inorganic nanoparticle layer comprising the inorganic nanoparticles coated with a polymer for coating inorganic nanoparticles and (b2) a polymer layer for coating capsules once or repeatedly on the surface of the hollow core polymer layer, and the polymer for coating inorganic nanoparticles are crosslinked.

Hereinafter, the first exemplary embodiment will be described.

As set forth above, in the hollow microcapsule according to the first exemplary embodiment, the organic-inorganic complex layer may be composed of one or a plurality of organic-inorganic complex layers formed by alternately stacking the (b1) inorganic nanoparticle layer comprising inorganic nanoparticles and the (b2) polymer layer for coating capsules comprising the polymer for coating capsules at least once on the surface of the hollow core polymer layer.

For example, the organic-inorganic complex layer may be formed by sequentially forming the (b1) inorganic nanoparticle layer comprising inorganic nanoparticles and the (b2) polymer layer for coating capsules comprising the polymer for coating capsules on the surface of the hollow core polymer layer. Alternatively, the organic-inorganic complex layer may be formed by sequentially forming the (b1) inorganic nanoparticle layer, the (b2) polymer layer, the (b1) inorganic nanoparticle layer, and the (b2) polymer layer on the surface of the hollow core polymer layer.

According to one exemplary embodiment, the hollow core polymer layer may be (i) a single polymer core layer of a positively charged polymer, or (ii) a complex polymer core layer formed by alternately stacking a positively charged polymer layer and a negatively charged polymer layer at least once, and an outermost polymer layer of the complex polymer core layer may be a positively charged polymer layer.

The hollow core polymer layer may be (i) a single polymer core layer of a positively charged polymer. Alternatively, the hollow core polymer layer may be (ii) a complex polymer core layer formed by alternately stacking a positively charged polymer layer and a negatively charged polymer layer at least once. Particularly, the hollow core polymer layer of (ii) may have more advantageous effects than the hollow core polymer layer of (i) in that the surface of the sacrificial core is much smoother, thereby facilitating formation of the organic-inorganic complex layer.

It is possible to obtain a smooth surface by repeatedly coating either one of the polymers several times, instead of repeatedly coating the positively charged polymer and the negatively charged polymer. It can be confirmed that repeated coating of the positively charged polymer and the negatively charged polymer can easily accomplish a smooth surface in a layer-by-layer (LbL) manner, which enables formation of the organic-inorganic complex layer with high yield under milder conditions through alternate stacking of the inorganic nanoparticle layer and the polymer layer for coating capsules. Furthermore, it could also be confirmed that lamination of multiple layers in an LbL manner could increase mechanical properties and stability as compared to the single polymer layer.

In addition, in the case where the complex polymer core layer is a single layer, the single layer is preferably a single layer of the positively charged polymer. In the case where the complex polymer core layer comprises multiple layers, it is preferred that the outermost layer is a positively charged complex layer, which is beneficial for alternately stacking a negatively charged inorganic nanoparticle layer and a positively charged polymer layer in an LbL manner on the surface of the core polymer layer.

Furthermore, it can be confirmed that the complex polymer core layer having a thickness of 8 nm to 12 nm, preferably 9 nm to 11 nm, is advantageous in view of maintaining excellent stability under repeated severe elastic deformation.

According to another exemplary embodiment, the positively charged polymer may be selected from chitosan, polylysine, polyethyleneime (PEI), polyallylamine hydrochloride (PAH), polyallyldimethyl ammonium chloride (PDADMAC), and a mixture thereof, and the negatively charged polymer may be selected from alginate, heparin, polystyrene sulfonate (PSS), polyacrylic acid (PAA), and a mixture thereof.

According to a further exemplary embodiment, the hollow core polymer layer may be (i) a chitosan polymer core layer, or (ii) a complex polymer core layer formed by alternately stacking an alginate layer and a chitosan layer at least once on the hollow chitosan layer, and an outermost polymer layer of the complex polymer core layer may be the chitosan polymer layer.

According to yet another exemplary embodiment, the organic-inorganic complex layer (b) may be composed of 1 to 30 organic-inorganic complex layers of the inorganic nanoparticle layers and the polymer layers for coating capsules.

The organic-inorganic complex layer (b) may be composed of 1 to 30, preferably 2 to 10, more preferably 2 to 5 organic-inorganic complex layers of the inorganic nanoparticles and the polymer for coating capsules.

According to yet another exemplary embodiment, the inorganic nanoparticle may be selected from silica, hydroxyapatite, magnetite, gold, silver, and a mixture thereof.

In the present invention, hydroxyapatite may be capped with citrates and a mixture thereof, since such capping can significantly improve dispersion stability in water through negative charge repulsion. If such capping is not performed, it is necessary to perform an additional step such as sonication. Furthermore, capping advantageously allows rapid precipitation of non-capped nanoparticles, thereby facilitating the layer-by-layer process.

According to yet another exemplary embodiment, the polymer for coating capsules may be a positively charged polymer.

According to yet another exemplary embodiment, the (b) organic-inorganic complex layer may be selected from a complex layer formed by sequentially stacking 1 to 10 layers of silica layers and chitosan layers, a complex layer formed by sequentially stacking 1 to 10 layers of hydroxyapatite layers and chitosan layers, and a complex layer formed by sequentially stacking 1 to 10 layers of magnetite layers and chitosan layers.

According to yet another exemplary embodiment, the hollow microcapsule may further include an outermost polymer layer on a surface of the outermost polymer layer for coating capsules.

According to yet another exemplary embodiment, the outermost polymer layer may be a negatively charged polymer layer.

By additional coating, the outermost polymer layer can be positively or negatively charged, thereby advantageously allowing the outermost polymer layer to have a charge opposite to that of an osmotic inducing polymer electrolyte used in osmotic pressure experiments, thereby facilitating the osmotic pressure experiments. For example, if negatively charged polystyrene sulfonate is used as an osmotic inducing polymer electrolyte, the outermost polymer layer can be advantageously positively charged.

Specifically, in the case where the outermost polymer layer is chitosan, crosslinking between particles may occur, thereby making it difficult to form uniform particles. As demonstrated in Example 6-1, Example 6-2, Example 7, and Example 8, in the case where the outermost layer is an alginate layer instead of a chitosan layer, particles are not agglomerated together, thereby preventing crosslinking therebetween.

Hereinafter, the second exemplary embodiment will be described.

According to the second exemplary embodiment, the organic-inorganic complex layer may be composed of one or a plurality of organic-inorganic complex layers formed by alternately stacking the (b1′) inorganic nanoparticle layer comprising the inorganic nanoparticles coated with a polymer for coating inorganic nanoparticles and the (b2) polymer layer for coating capsules once or plural times repeatedly on the surface of the hollow core polymer layer, and the polymer for coating inorganic nanoparticles may be crosslinked.

For example, the organic-inorganic complex layer may be formed by sequentially forming the (b1′) coated inorganic nanoparticle layer comprising the inorganic nanoparticles coated with a polymer for coating inorganic nanoparticles and the (b2) polymer layer for coating capsules comprising the polymer for coating capsules on the surface of the hollow core polymer layer. Alternatively, the organic-inorganic complex layer may be formed by sequentially forming the (b1′) coated inorganic nanoparticle layer, the (b2) polymer layer for coating capsules, the (b1′) coated inorganic nanoparticle layer, and the (b2) polymer layer for coating capsules on the surface of the hollow core polymer layer.

Herein, the term “coated inorganic nanoparticle layer” may refer to an inorganic nanoparticle layer coated with a “polymer”.

According to one exemplary embodiment, the hollow core polymer layer may be (i) a single polymer core layer of a negatively charged polymer, or (ii) a complex polymer core layer formed by alternately stacking a positively charged polymer layer and a negatively charged polymer layer at least once, and an outermost polymer layer of the complex polymer core layer is a negatively charged polymer layer.

The hollow core polymer layer may be (i) a single polymer core layer of a negatively charged polymer. Alternatively, the hollow core polymer layer may be (ii) a complex polymer core layer formed by alternately stacking a positively charged polymer layer and a negatively charged polymer layer at least once.

Here, it should be understood that a smooth surface can be formed by repeatedly coating either one of the polymers several times, instead of repeatedly coating the positively charged polymer and the negatively charged polymer. It is confirmed that repeated coating of the positively charged polymer and the negatively charged polymer can easily form a smooth surface by a layer-by-layer (LbL) method, thereby facilitating formation of the organic-inorganic complex layer under milder conditions with high yield through alternate stacking of the inorganic nanoparticle layer and the polymer layer for coating capsules.

In addition, if the complex polymer core layer is a single layer, the single layer is preferably a single layer of the negatively charged polymer. If the complex polymer core layer comprises multiple layers, the outermost layer is preferably a negatively charged complex layer, which is advantageous for stacking a coated inorganic nanoparticle layer in an LbL manner on the surface of the core polymer layer. That is, since typical inorganic nanoparticles are negatively charged and are coated with positively charged polymers, the coated nanoparticle layer stacked in the LbL manner on the surface of the core polymer layer is positively charged.

Furthermore, it is confirmed that the complex polymer core layer having a thickness of 8 to 12 nm, preferably 9 to 11 nm is advantageous in view of maintaining excellent stability under repeated severe elastic deformation.

According to another exemplary embodiment, the positively charged polymer may be selected from chitosan, polylysine, polyethyleneime (PEI), polyallylamine hydrochloride (PAH), polyallyldimethyl ammonium chloride (PDADMAC), and a mixture thereof; and the negatively charged polymer may be selected from alginate, heparin, polystyrene sulfonate (PSS), polyacrylic acid (PAA), and a mixture thereof.

According to a further exemplary embodiment, the hollow core polymer layer may be a single alginate layer.

According to yet another exemplary embodiment, the (b) organic-inorganic complex layer may be composed of 1 to 30 organic-inorganic complex layers of the coated inorganic nanoparticle layers and the polymer layers for coating capsules.

The (b) organic-inorganic complex layer may be composed of 1 to 30, preferably 2 to 10, more preferably 2 to 5 organic-inorganic complex layers of the coated inorganic nanoparticles and the polymer for coating capsules.

According to yet another exemplary embodiment, the inorganic nanoparticles may be selected from silica, hydroxyapatite, magnetite, gold, silver, and a mixture thereof.

In the present invention, hydroxyapatite is preferably capped with citrates, and a mixture thereof since such capping can significantly improve dispersion stability in water through negative charge repulsion.

According to yet another exemplary embodiment, the polymer for coating inorganic nanoparticles may be a positively charged polymer and the polymer for coating capsules may be a negatively charged polymer.

According to yet another exemplary embodiment, the (b) organic-inorganic complex layer may be a complex layer formed by sequentially stacking 1 to 10 layers of chitosan coated silica layers and alginate layers.

According to yet another exemplary embodiment, the hollow microcapsule may further include an outermost polymer layer on a surface of the outermost polymer layer for coating capsules, and the outermost polymer layer may be a positively charged polymer layer.

A further aspect of the present invention relates to a scaffold for soft tissue including a hollow microcapsule according to various embodiments of the present invention.

Yet another aspect of the present invention relates to a drug delivery carrier including a hollow microcapsule according to various embodiments of the present invention.

According to one embodiment, the drug delivery carrier may respond to mechanical stimuli or may be controllable by mechanical stimuli.

Yet another aspect of the present invention relates to a method of preparing a hollow microcapsule including: (A) forming a core polymer layer on \textcircled{1} a positively charged sacrificial core or \textcircled{2} a negative charge-modified sacrificial core; (B) \textcircled{1} if the sacrificial core is the positively charged sacrificial core, alternately forming an inorganic nanoparticle layer and a polymer layer for coating capsules at least once on the core polymer layer, and \textcircled{2} if the sacrificial core is the negative charge-modified sacrificial core, alternately forming an inorganic nanoparticle layer coated with a composition for coating inorganic nanoparticles and a polymer layer for coating capsules at least once on the core polymer layer; (C) crosslinking the core polymer and the polymer for coating capsules; and (D) removing the sacrificial core by etching.

According to one embodiment, the positively charged sacrificial core may be a calcium carbonate micro-particle, the negative charge-modified sacrificial core may be a calcium carbonate micro-particle modified with phosphate, and the core polymer layer and the polymer layer for coating capsules may be formed by a layer-by-layer method.

In the present invention, modification using phosphate may be performed by bringing calcium carbonate into contact with a Na₂HPO₄ solution having a pH of 9 to 11.

In addition, chitosan may be crosslinked using a crosslinking agent such as glutaraldehyde, and alginate may be crosslinked using Ca²⁺ ions.

Preferably, (C) crosslinking is performed at subzero temperature. More preferably, (C) crosslinking is performed when the polymer to be crosslinked is chitosan. In this case, it is confirmed that the crosslinked bonds have significantly increased flexibility and the polymer layer has remarkably enhanced elasticity.

Namely, the hollow microcapsule according to the present invention can have elasticity to be deformed in application of external force thereto and to be recovered to an original shape thereof when the external force is removed therefrom.

In addition, the hollow microcapsule can maintain properties of deformation and recovery after repeated application and removal of external force.

The present invention will be described in more detail with reference to the following examples. However, it should be understood that the following examples should be interpreted as illustrative and not in a limiting sense. Further, it is apparent to those skilled in the art that the present invention, concrete experimental results of which are not disclosed, can be easily realized by those skilled in the art, only if the present invention is based on the disclosure of the present invention including the following examples. Naturally, any variants and modifications fall within the scope of the appended claims.

According to various examples of the present invention, a method of preparing a bioabsorbable, biocompatible, elastic and macroporous hydroxyapatite-gelatin hybrid scaffold which could be resiliently recovered after about 90% deformation from initial shape wherein HAp amount was 95% at maximum was provided. Gelatin coated HAp particles were crosslinked with EDC, followed by lyophilization at −5° C. to −80° C. to obtain a scaffold of a porous structure. Elastic properties of the prepared scaffold were irrelevant to those of the used particles, as proved by scaffolds prepared using PLGA nanospheres. Materials having different compressive elasticity were prepared by changing EDC concentrations and particle amounts. Biocompatibility of these scaffolds was confirmed through in vitro and in vivo experiments.

In addition, the present invention provides a method of synthesizing crosslinked hybrid silica nanoparticles/biocompatible polymer hollow microcapsules through freezing, which demonstrates a maximum elastic deformation recovery of 90%. Capsules were prepared by alternately adsorbing chitosan particles and 7 nm colloidal silica particles on calcium carbonate micro-particles which can be etched by an EDTA solution, wherein the chitosan layer was crosslinked using glutaraldehyde.

In the method of preparing elastic scaffolds according to the present invention, hydroxyapatite, silica or PLGA nanoparticles are used as a biocompatible main component in amounts of up to 95%; gelatin, chitosan or heparin is used as a biopolymer for coating these particles; and an element selected from telechelic diepoxy, glutaraldehyde, 1-ethyl-3-(3-dimethylaminopropyl)carboimide (EDC), N,N-carbonyl diimidazole (CDI) and a mixture thereof is used as a crosslinking agent.

Furthermore, the hollow capsule according to the present invention is prepared using silica, hydroxyapatite or magnetite nanoparticles as an inorganic component, chitosan, gelatin or alginate as a polymer component, glutaraldehyde or telechelic diepoxy as a crosslinking agent, and calcium carbonate as a sacrificial core.

### EXAMPLE

### Example 1

### Preparation of Hydroxyapatite/Gelatin Scaffold Crosslinked Using EDC as a Crosslinking Agent and Examination of Properties (Citrate-Capped HAp @ EDC-Crosslinked Gelatin)

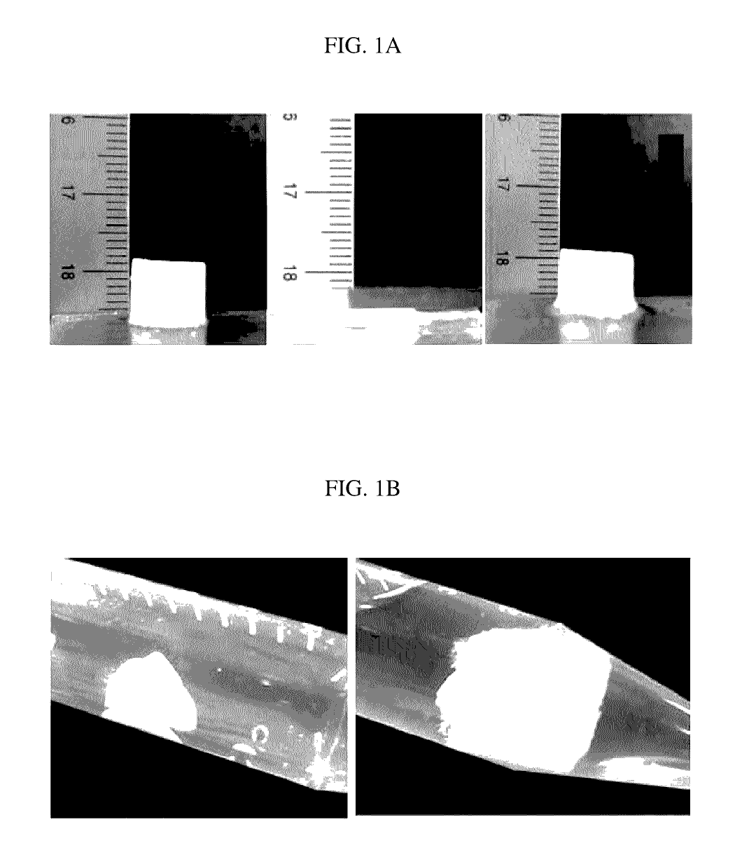

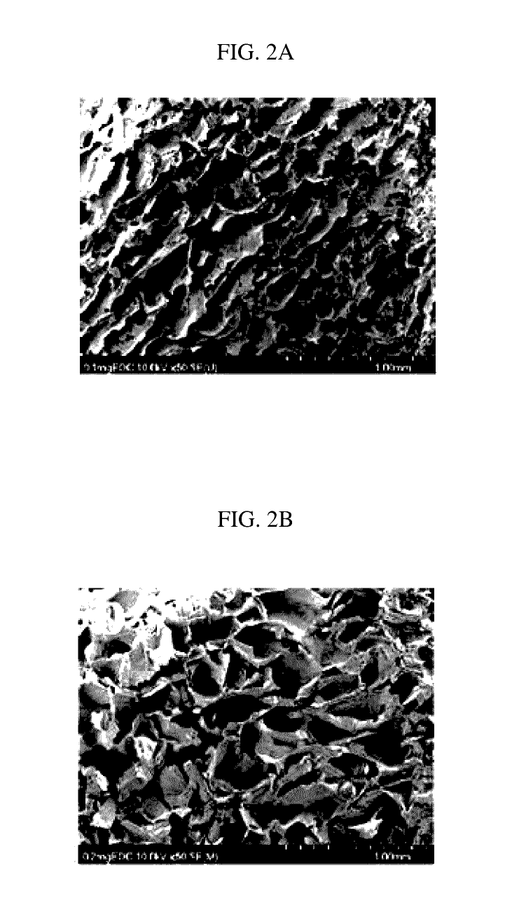

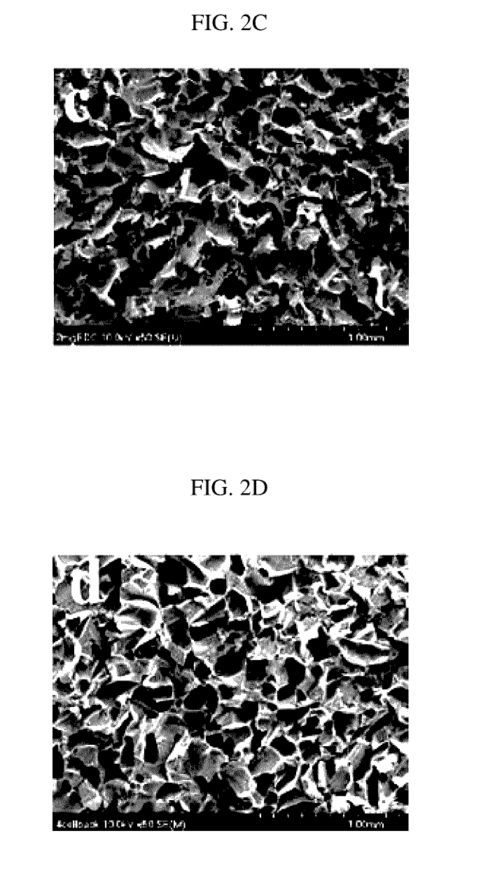

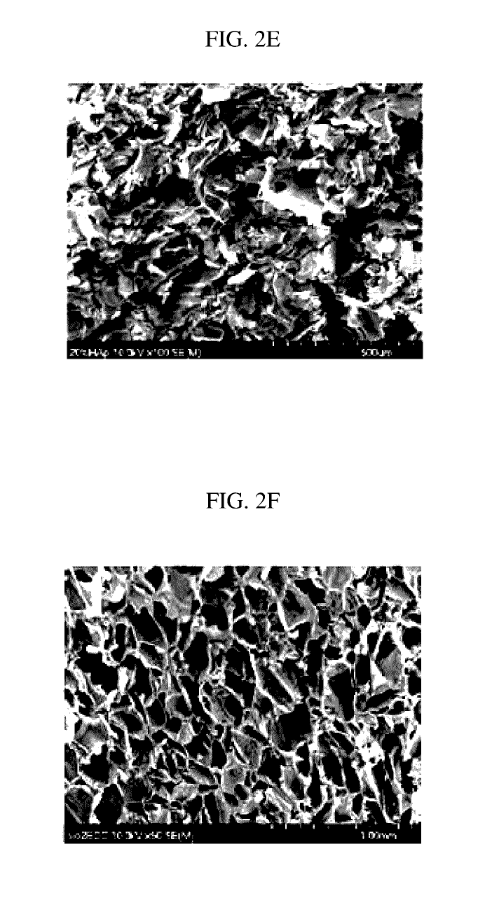

Hydroxyapatite nanoparticles capped with citrates and coated with gelatin (porcine derived B type gelatin) in a size of ˜200 nm were crosslinked at −18° C. to prepare soft and resiliently recoverable macroporous hydroxyapatite/gelatin scaffolds. The final solution prior to freezing was maintained such that a weight ratio of a polymer to particles was 1:10. Namely, 60 mg of particles were coated with 6 mg of gelatin in 0.6 mL deionized water, and the amount of EDC was changed to 0.1 mg, 0.5 mg, 2 mg and 4 mg (SEM images of FIGS. 2A to 2D SEM). FIG. 1A is a digital image of 4 mg EDC scaffolds, which clearly shows that the scaffolds recovered their shape after high compressive strain. The particles were intensely mixed with gelatin, stirred and coated, and EDC was added thereto as a crosslinking agent prior to freezing. The crosslinking density of the polymers exerted a strong effect on mechanical properties of the prepared scaffolds. When the crosslinking density of the polymer was the lowest value due to use of 0.1 mg of EDC, very soft jellylike scaffolds were obtained and maintained a complete shape in a solvent (water) (FIG. 1B). Identical results were found even if the concentration of gelatin was lowered to 3 mg based on 60 mg of particles in 0.6 mL deionized water. In addition, an appropriate concentration of gelatin in the final solution to obtain scaffolds having suitable strength was 1 wt %.

Porosity of scaffolds can be adjusted by changing a freezing temperature in the range of −5° C. to −80° C. and the amount of particles in the final solution. Crosslinking time at all temperatures was 24 hours. As the amount of particles increased, porosity decreased, but mechanical strength of scaffolds increased. The same behavior was observed when scaffolds were prepared by changing the concentration of hydroxyapatite particles to 20 wt % (120 mg in 0.6 mL of deionized water), the concentration of gelatin to 1% (6 mg in 0.6 mL of deionzied water), and the concentration of EDC to 4 mg in 0.6 mL of the final solution (FIG. 2E).

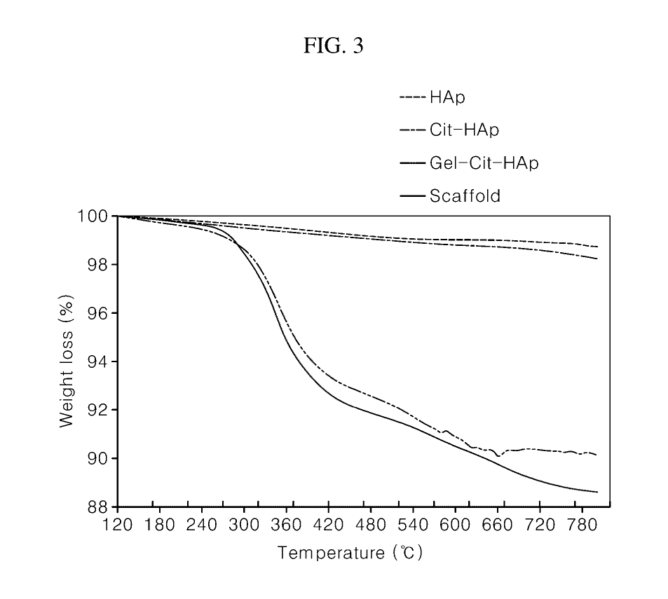

Thermogravimetric analysis (TGA) for scaffolds comprising untreated hydroxyapatite particles, citrate capped hydroxyapatite particles, gelatin-coated hydroxyapatite particles, and 10% hydroxyapatite/1% gelatin/4 mg EDC was performed. For analysis, scaffolds in a thin disk shape were used after lyophilization. As a result, the scaffolds were composed of 90% of inorganic materials and 10% of organic materials (FIG. 3).

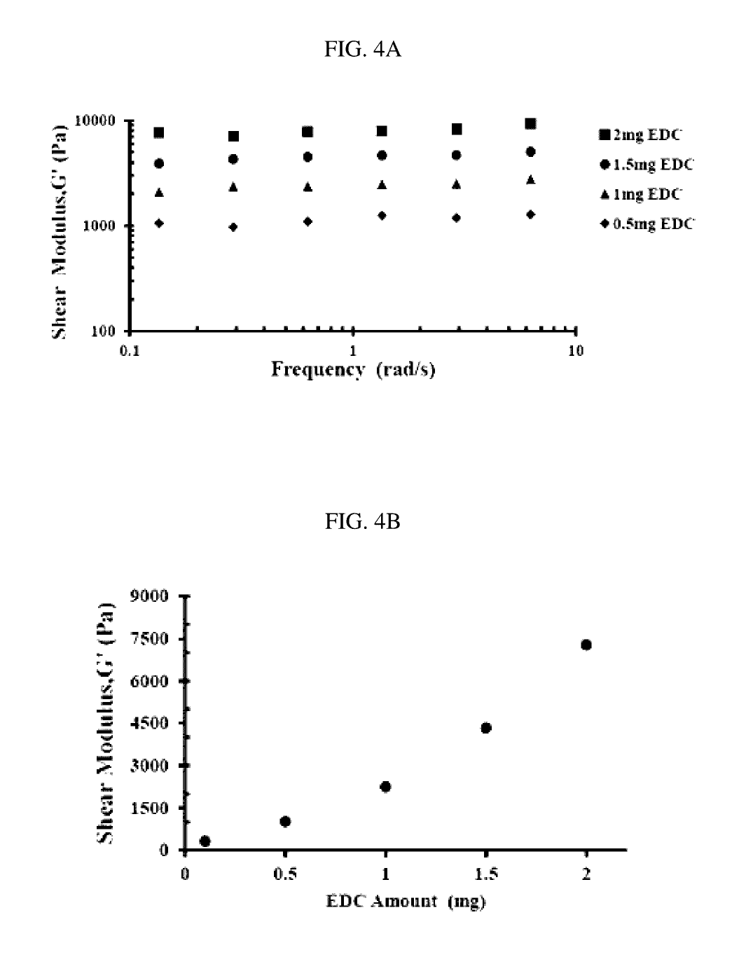

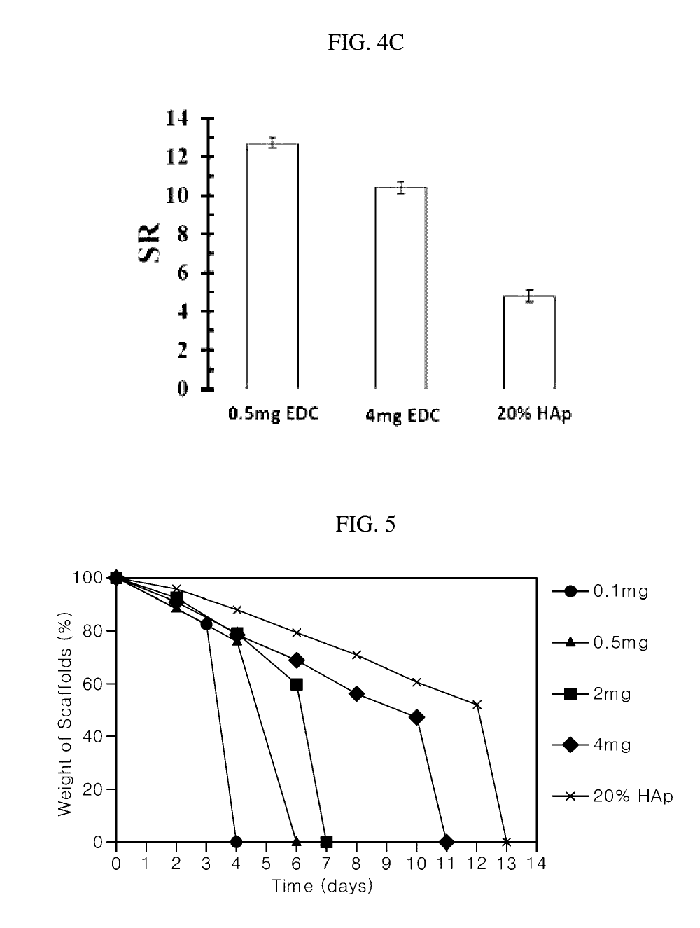

Scaffold disks having a height of 2 mm and a diameter of 8 mm were subjected to rheological analysis. In order to induce linear vibration deformation, the frequency was set to ω=10 rad/s and the deformation rate was set to γ=0.025%. In all experiments, values of ω and γ were constant. Shear modulus of scaffolds was 300 Pa for scaffolds comprising 0.1 mg EDC, and 7 kPa for scaffolds comprising 2 mg EDC. Shear modulus increased with increasing crosslinking density (FIG. 4A and FIG. 4B).

The swelling rate of scaffolds was measured by gravimetric analysis. Lyophilized HAp scaffold was weighed and dipped in deionized water for 5 minutes. Water on the surface of swollen samples was wiped off with filter paper, followed by weighing, and the swelling rate (SR) of the scaffold was calculated from Equation (1):

SR=(Wh−Wd)/Wd (1),

wherein Wh is an equilibrium weight of swollen scaffolds, and Wd is a weight of dried scaffolds. Calculation was made using 4 identical samples three times and the calculated values were averaged.

The swelling rate and mechanical properties of scaffolds may be controlled by two parameters. Firstly, the crosslinking density of scaffolds may be adjusted by changing the EDC amount prior to lyophilization. Secondly, the concentration of slurries of particles may be adjusted by changing the amounts of gelatin and EDC. In the case where scaffolds comprise only polymers, the latter is not applied. This can be confirmed from rheological data depicted in FIG. 4A and FIG. 4B. The mechanical properties of scaffolds prepared under certain conditions using such properties can determine applications of the scaffolds. For example, since scaffolds comprising 0.5 mg of EDC have a modulus of 3 kPa similar to adipose tissue, such scaffolds can be used in tissue engineering of adipose tissue. As expected, the swelling rates and storage moduli of scaffolds decreased with increasing crosslinking density (FIG. 4C).

Disk-shaped HAp-Gel scaffolds subjected to washing, autoclaving and lyophilization were examined for decomposition properties. Samples were cut into a diameter of 8 mm and a thickness of 1.5 to 2 mm, and then weighed. Scaffolds comprising 10% gelatin alone were used as control groups. Crosslinked gelatin was subjected to enzyme decomposition in the presence of collagenases in a phosphate buffer solution (PBS). The enzyme solution was prepared using 0.16 mg/mL of PBS (1%, pH 7.4), Clostridium histolyticum-derived collagenase, 1.45 mg/mL of calcium chloride-PBS solution as an activator, and 0.01 mg/mL (0.001%) of sodium azide as an antibacterial agent. Each of scaffolds having different crosslinking densities was dipped in 1.5 mL of an enzyme solution on a 48-well tissue culture dish, and maintained at 37° C. in an incubator. Time taken to enzymatically decompose scaffolds in vitro increased with increasing crosslinking density. Under all conditions, decomposition was completed within 2 weeks (FIG. 5). For scaffolds comprising 20% HAp, since enzymes were required to pass through dense walls comprising particle networks, decomposition took considerably time.

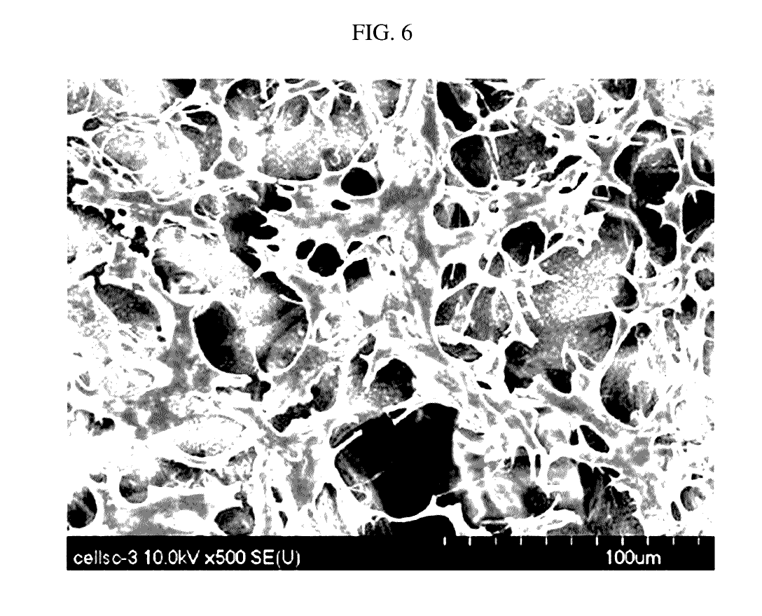

NIH 3T3 fibroblast cells were cultured in a complete medium DMEM-F12 (Dulbecco's Modified Eagle Medium Nutrient Mixture F-12) supplemented with 10% fetal bovine serum and 1% antibiotic solution at 37° C. in a 5% CO₂ atmosphere. The medium was exchanged every 48 hours. After cells were harvested from the culture dish using 0.25% trypsin, about 100 μl of cell suspension containing 5×10⁵ cells was plated onto a lyophilized and sterilized scaffold. The scaffold was incubated for 1 hour and dipped in a complete medium solution. FIG. 6 is an SEM image taken after incubating a scaffold for three days wherein the scaffold comprising 10% hydroxyapatite/1% gelatin/4 mg EDC was plated with NIH 3T3 cells in order to identify biocompatibility of the scaffold. From the SEM image, it could be seen that cells were well attached to walls of the scaffold.

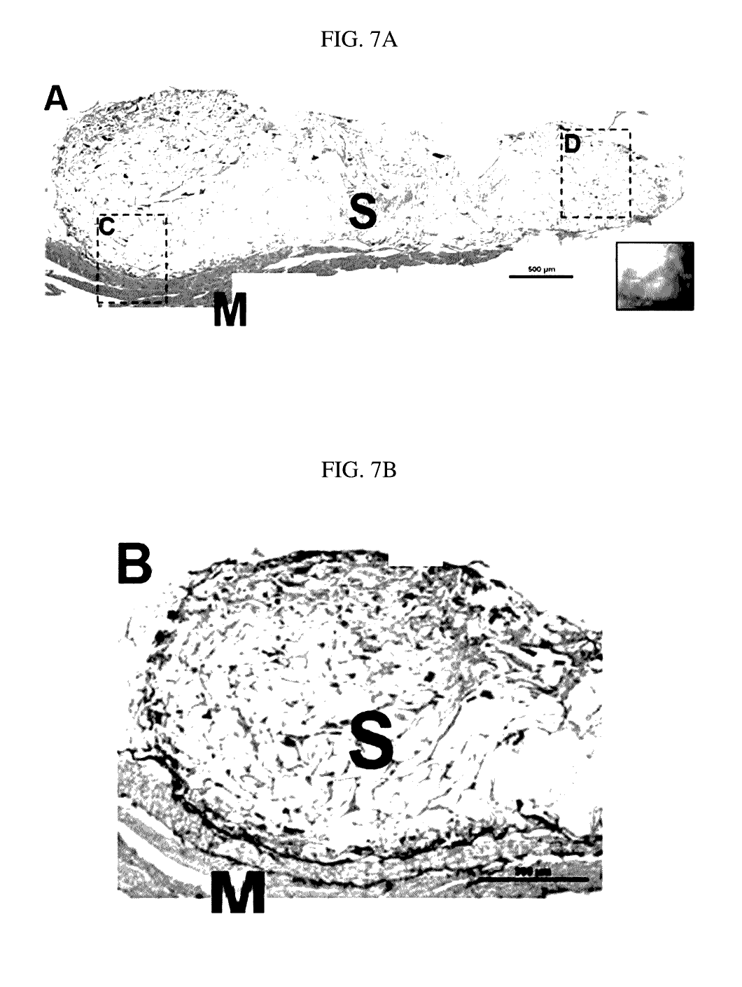

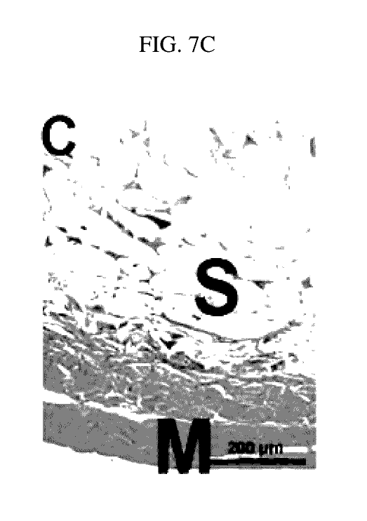

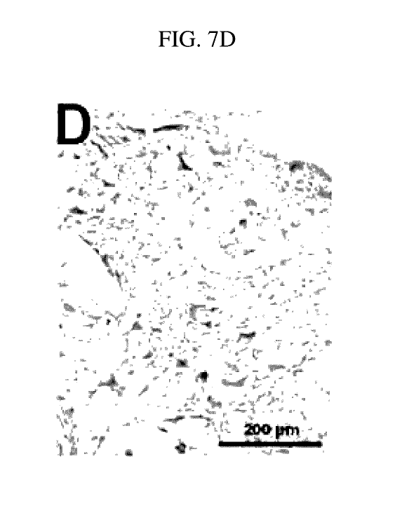

The synthesized scaffold was washed with deionized water and then heated in an autoclave. The sterilized scaffold was pretreated under physiological conditions in the cell culture medium (DMEM, Sigma-Aldrich, Mo., USA). The resulting scaffold was lyophilized under aseptic conditions. Animal experiments were performed under recognition of the Institutional Animal Care and Use Committee of Kwangju Institute of Science and Technology (GIST). Male mice (Balb/c, five month, Orientbio Co., Ltd., Kyenggi Province, Korea) were anesthetized with isoflurane and transplanted with the sterilized scaffold in a subcutaneous space. After two weeks, these mice were sacrificed, thereby recovering the scaffold. The recovered sample was fixed in a formaldehyde solution and then embedded in paraffin. The scaffold in the paraffin block was sliced into a thickness of 6 μm using a microtome (Leica RM2135, Wetzlar, Germany) Sample slides were stained with hematoxylin-eosin and Sirius red, and then observed through a brightfield microscope (Axioskop40, Carl Zeiss, Jena, Germany). As can be seen from FIGS. 7A and 7B, scaffolds transplanted for two weeks were found to be surrounded with a very thin collagen layer and a few immune cells were on the boundary of the scaffold and the living body. In addition, a plurality of blood vessels was observed in the scaffold, which confirmed that a considerable amount of tissues from the living body grew into the transplanted scaffold. Accordingly, it was confirmed that the scaffold had excellent biocompatibility in vivo.

### Example 2

### Preparation of Crosslinked Silica/Gelatin Scaffold Using EDC Crosslinking Agent (Silica @ EDC-Crosslinked Gelatin)

10% by weight of silica nanoparticles having a size of 500 nm were vortexed in an e-tube such that the nanoparticles were coated with 1% gelatin. The volume of the final solution was 0.6 mL wherein amounts of particles and polymers were 60 mg and 6 mg, respectively. 4 mg of an EDC crosslinking agent was added to the final solution, followed by freezing at −18° C. for 24 hours to complete crosslinking. Mechanical properties of the obtained scaffold were similar to those of the scaffold obtained in Example 1 comprising 10% hydroxyapatite/1% gelatin/4 mg EDC (FIG. 2F). Walls of the scaffold mainly consisted of silica particles.

### Example 3

### Preparation of Scaffolds Comprising Crosslinked PLGA/Gelatin Using EDC Crosslinking Agent (PLGA @ EDC-Crosslinked Gelatin)

PLGA nanoparticles having a size of about 500 nm were synthesized by solvent emulsification. In order to improve stability of a PLGA suspension in water, the obtained particles were coated with gelatin. A suspension of the coated particles was heated to 45° C. to increase stability thereof. Weight ratio of particles to polymers was 10:1. 0.6 mL of the final deionized suspension had EDC in amounts of 4 mg. Crosslinking was performed at −25° C. for 24 hours.

### Example 4

### Preparation of Scaffolds Comprising Crosslinked Hydroxyapatite/Chitosan Using Telechelic Diepoxy Crosslinking Agent (Citrate-Capped HAp @ TKD-Crosslinked Chitosan)

10% by weight of citrate-capped hydroxyapatite nanoparticles in a size of about 200 nm were vortexed in an e-tube such that the nanoparticles were coated with 1% gelatin. The volume of the final solution was 0.6 mL wherein amounts of particles and polymers were 60 mg and 6 mg, respectively. 5 mg of a telechelic diepoxy crosslinking agent was added to the final solution, followed by freezing at −18° C. for 24 hours to complete crosslinking.

### Example 5

### Preparation of Scaffolds Comprising Crosslinked Hydroxyapatite/Chitosan Using Glutaraldehyde Crosslinking Agent (Citrate-Capped HAp @ GA-Crosslinked Chitosan)

10% by weight of citrate-capped hydroxyapatite nanoparticles having a size of about 200 nm were vortexed in an e-tube such that the nanoparticles were coated with 1% chitosan. The volume of the final solution was 0.6 mL wherein amounts of particles and polymers were 60 mg and 6 mg, respectively. 5 mg of a glutaraldehyde crosslinking agent was added to the final solution, followed by freezing at −18° C. for 24 hours to complete crosslinking.

### Example 6

### Preparation of Silica/Chitosan Hybrid Hollow Capsule Using Calcium Carbonate Particles as Templates

A hollow capsule was prepared using calcium carbonate micro-particles as a sacrificial core in accordance with a reported method. Spherical calcium carbonate particles having an average particle diameter of 6 μm to 20 μm were synthesized through simple precipitation. A sodium carbonate solution and a calcium chloride solution having the same molar concentration and volume were rapidly mixed, and stirred at 1,000 RPM in a 100 mL round-bottom flask. The size of CaCO₃ core could be adjusted by changing reaction time and concentrations of reactants. The core was insoluble at pH 7 and completely dissolved at acidic pH, namely, pH≦4.

A hybrid hollow capsule was prepared using two different coating methods. In the first coating method, chitosan and 7 nm Ludox SM colloidal silica particles were alternately coated onto spherical calcium carbonate sacrificial particles. In the second coating method, 7 nm Ludox SM colloidal silica particles coated with chitosan and alginate were alternately coated onto modified spherical calcium carbonate sacrificial particles.

(1) The First Method (\textcircled{1} Phosphate Modified CaCO₃ @ Chi-Alg-Chi-(SiO₂-Chi)₃-Alg)

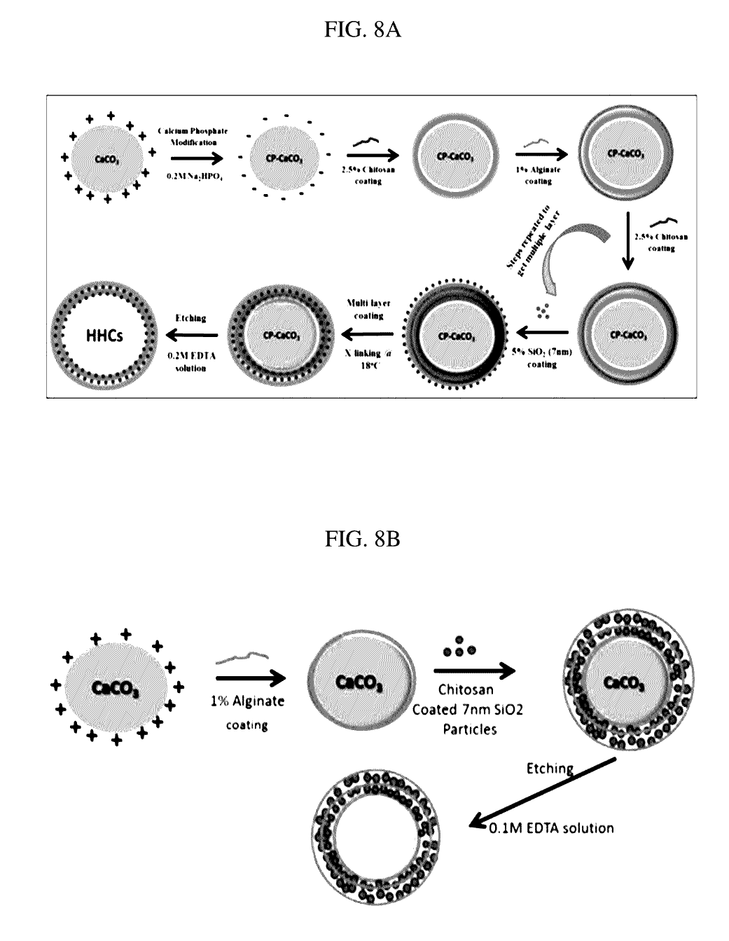

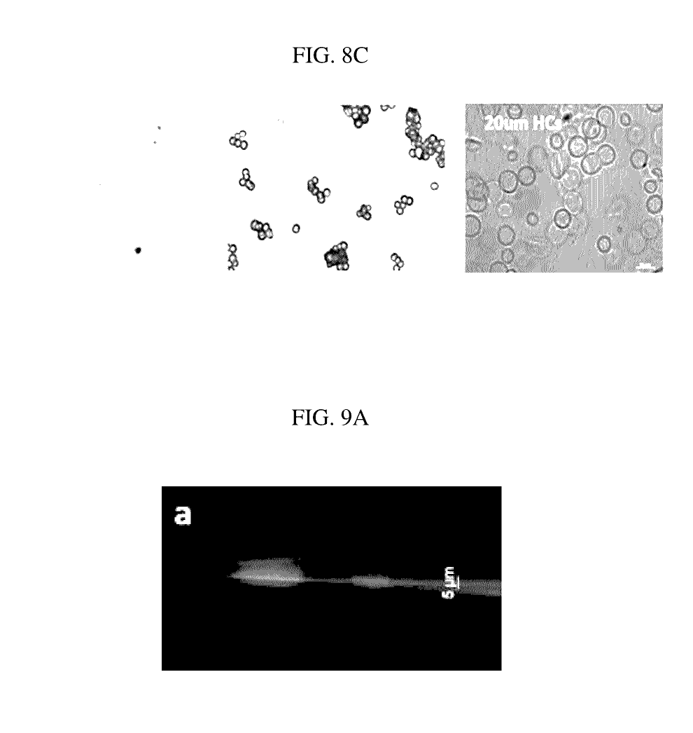

As depicted in FIG. 8A, calcium carbonate particles were reacted with 0.2 M Na₂HPO₄ at pH 10 (pH was adjusted using NaOH solution), thereby modifying surfaces of the calcium carbonate particles with phosphate ions. Prior to full-scale polymer coating, a polymer base consisting of chitosan-alginate-chitosan was formed as follows.

A modified calcium carbonate core having a certain weight was dispersed in deionized water, followed by ultrasonification for 10 minutes, and mixed with a 5% chitosan solution in a 0.5M NaCl solution for 10 minutes. Thereafter, the core was mixed with a 1% alginate solution in a 0.5M NaCl solution for 10 minutes, thereby coating the core with alginate. The alginate-coated CaCO₃ was mixed with a 5% chitosan solution in a 0.5 M NaCl solution for 10 minutes, thereby coating the core with chitosan. Chi-Alg-Chi coated (coating order in the present invention is represented from left layer to right layer) CaCO₃ particles were mixed with 2.5% 7 nm Ludox SM colloidal silica particles for 10 minutes, thereby coating a 7 nm Ludox SM colloidal silica particle layer as a fourth layer. A chitosan layer as a fifth layer was coated onto the core in the same manner as above. After each step, the core was washed with 0.1 M NaCl three times. The fourth and fifth layers were repeated to form layers of desired numbers.

(2) The second method (\textcircled{2} CaCO₃ @ Alg-(Chi @ SiO₂-Alg)₃)

As shown in FIG. 8B, non-modified CaCO₃ particles were used as a sacrificial core. Alginate coated CaCO₃ was mixed with a dispersion of chitosan coated 7 nm Ludox SM colloidal silica particles for 10 minutes, thereby forming a chitosan coated 7 nm Ludox SM colloidal silica particle layer as the second layer.

Alg-Chi@SiO₂ coated (Chi@SiO₂ refers to chitosan coated silica particles) CaCO₃ particles were mixed with 1% sodium alginate for 10 minutes, thereby forming an alginate layer as the third layer. After each step, the core was washed with 0.5 M NaCl three times. The fourth and fifth layers were repeated to form layers of desired numbers. In both methods, alginate was used as a final layer in order to inhibit agglomeration.

(3) Crosslinking and etching (\textcircled{1} Chi-Alg-Chi-(SiO₂-Chi)₃-Alg, \textcircled{2} Alg-(Chi @ SiO₂-Alg)₃)

In both cases, crosslinking was performed as follows. Capsule particles with multiple layers prepared by these two methods were mixed with 200 μL of a 50% glutaraldehyde solution, followed by lyophilizing at −18° C. and crosslinking for 24 hours.

After completion of crosslinking, the particles were washed with water and CaCO₃ three times, and etched with a 0.1 M EDTA solution at pH 7.5 for 3 hours.

### (4) Elastic Behavior Observation

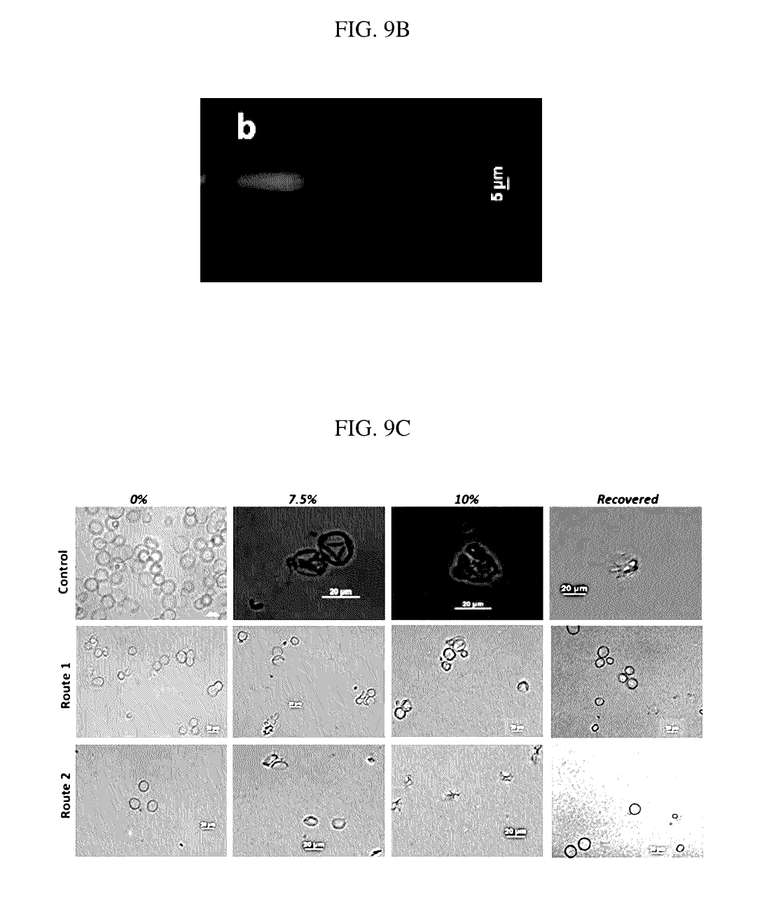

Hybrid hollow capsules (HHCs) having various sizes were obtained using calcium carbonate cores having different sizes by an almost identical method (FIG. 8B). After pressing the capsules (HHCs) through a patch clamp, an inner diameter of which was 80% smaller than that of capsules, deformation and recovery were measured to identify elastic behaviors (FIG. 9A and FIG. 9B). The capsules were completely recovered after deformation to 80% to 90%. HHCs exhibited recovery properties after deformation by osmotic pressure, whereas control capsules not including particles in shells were broken when osmotic pressure was applied thereto (FIG. 9C). The osmotic pressure experiment was performed by incubating capsules having a chitosan layer as the final layer in a poly(styrene sulfonate) (PSS, Mw 70 kDa) solution in various concentrations for 10 minutes.

### Example 7

### Preparation of Hydroxyapatite/Chitosan Hybrid Hollow Capsules (Phosphate Modified CaCO₃ @ Chi-Alg-Chi-(Citrate-Capped HAp-Chi)₃-Alg)

Hydroxyapatite particles were purchased from Sigma Aldrich, and treated with 0.2M trisodium citrate at pH 6, which was adjusted with 0.1 M HCl, at room temperature for 12 hours. The particles were completely washed with deionized water. It was confirmed that the particles had an average particle diameter of 150 nm and zeta potential was −27 mV.

CaCO₃ particles were reacted with 0.2 M Na₂HPO₄ at pH 10 (pH was adjusted using NaOH) for 2 hours, thereby modifying surfaces of the CaCO₃ particles with phosphate. Prior to full scale polymer coating, a polymer base consisting of three layers of chitosan-alginate-chitosan was formed on the negatively charged phosphate modified particles as follows.

A modified calcium carbonate core having a certain weight was dispersed in deionized water, followed by ultrasonification for 10 minutes, and mixed with a 5% chitosan solution in a 0.5M NaCl solution for 10 minutes. Thereafter, the core was mixed with a 1% alginate solution in a 0.5M NaCl solution for 10 minutes, thereby coating the core with alginate. The alginate-coated CaCO₃ was mixed with a 5% chitosan solution in a 0.5 M NaCl solution for 10 minutes, thereby coating the core with chitosan.

Chi-Alg-Chi coated (coating order in the present invention is represented from left layer to right layer) CaCO₃ particles were mixed with 2.5% HAp particles for 10 minutes, thereby forming citrate-capped hydroxyapatite particles (average diameter 150 nm) as a fourth layer. A chitosan layer as a fifth layer was coated on the core in the same manner as above. After each step, the core was washed with 0.1 M NaCl three times. The fourth and fifth layers were repeated to form desired numbers of layers.

Crosslinking was performed as follows. CaCO₃ particles with multiple layers were mixed with 200 μL of a 50% glutaraldehyde solution, followed by lyophilizing at −18° C. and then crosslinked for 24 hours. After completion of crosslinking, the particles were washed with water and CaCO₃ three times, etched with 0.1 M EDTA solution of pH 5.5 for three hours.

### Example 8

### Preparation of Fe₃O₄/Chitosan Hybrid Hollow Capsules (Phosphate Modified CaCO3 @ Chi-Alg-Chi-(Magnetite-Chi)3-Alg)

pH values of an FeCl₃.6H₂O (0.1 M) solution and an FeCl₃.4H₂O (0.2 M) solution were adjusted using 1 M HCl to be acidic pH, to which 5% SDS surfactant was added to control agglomeration of particles. To this mixed solution, ammonium hydroxide was added under inactive ambient conditions until pH reached pH 12. The synthesized particles were washed with butyl alcohol, mixed with lauric acid and magnetic particles (ratio of 3:2) at 600° C. to coat surfaces of the particles with lauric acid. Uncoated lauric acid was washed with acetone, and resuspended in water using surfactants.

CaCO₃ particles were reacted with 0.2 M Na₂HPO₄ at pH 10 (pH was adjusted using NaOH) for 2 hours, thereby modifying surfaces of the CaCO3 particles with phosphate. Prior to full-scale polymer coating, a polymer base consisting of three layers of chitosan-alginate-chitosan was formed on the negatively charged phosphate modified particles as follows.

A modified calcium carbonate core having a certain weight was dispersed in deionized water, followed by ultrasonification for 10 minutes, and mixed with a 5% chitosan solution in a 0.5M NaCl solution for 10 minutes. Thereafter, the core was mixed with a 1% alginate solution in a 0.5M NaCl solution for 10 minutes, thereby coating the core with alginate. The alginate-coated CaCO3 was mixed with a 5% chitosan solution in a 0.5 M NaCl solution for 10 minutes, thereby coating the core with chitosan.

Chi-Alg-Chi coated (coating order in the present invention is represented from left layer to right layer) CaCO3 particles were mixed with 2.5% ferric oxide nanoparticles for 10 minutes, thereby forming ferric oxide magnetic nanoparticles (average diameter 15 nm) as a fourth layer. A chitosan layer as a fifth layer was coated onto the core in the same manner as in above. After each step, the core was washed with 0.1 M NaCl three times. The fourth and fifth layers were repeated to form desired numbers of layers.

Crosslinking was performed as follows. CaCO3 particles with multiple layers were mixed with 200 μL of a 50% glutaraldehyde solution, followed by lyophilizing at −18° C. and then crosslinking for 24 hours. After the completion of crosslinking, the particles were washed with water and CaCO₃ three times, and etched with a pH 5.5, 0.1 M EDTA solution for three hours.

### Example 9

### Preparation of Drug Delivery Carriers and Experiment to Measure Properties Thereof

(1) Preparation of Hollow Capsules

A single layered hybrid hollow capsule (1L-HHC) having a structure of (Chi-Alg-Chi)-(SiO₂-Chi)₁-Alg and a three-layer hybrid hollow capsule (3L-HHC) having a structure of (Chi-Alg-Chi)-(SiO₂-Chi)₃-Alg, respectively, were prepared in accordance with the first method disclosed in Example 6. For comparison, a three-layer hollow capsule (3L-HC) without inorganic nanoparticles having a structure of (Chi-Alg-Chi)-(Alg-Chi)₃-Alg was also prepared.

(2) Experiment for Loading Drugs in the Hollow Capsules and Releasing the Drug

The prepared hollow capsules were dispersed in a 0.1 M NaCl solution in which a model drug was dispersed, and stood at room temperature for 12 hours, thereby loading the drug in the hollow capsules. Drugs with various molecular weights such as FITC, PEI 800 Mw, PEI 1300 Mw, FITC-Dextran 4 kDa, Lysozyme 14 kDa, and FITC-BSA were used as the model drugs.

Onto a glass slide, surfaces of which were hydrophilized with Piranha solution (3:1, H₂O₂/H₂SO₄), a positively charged chitosan with Mw 70 kDa was coated, followed by coating the prepared drug loaded hollow capsules (negatively charged Alg was the outermost polymer layer). Pressure of 100, 250, and 500 g was applied manually for 6 seconds, and the released solution was harvested. The capsules were refilled with fresh water. Upon relieving pressure, the capsules having recovered from elastic deformation were stood for 10 minutes, and then a solution spread and released from the capsules was examined.

Amounts of drugs released from FITC, FITC-Dextran and FITC-BSA loaded capsules were analyzed through absorbance at 493 nm, and released amounts of PEI were analyzed by the Ninhydrin method at 570 nm, and released amounts of lysozyme were analyzed through absorbance at 275 nm-280 nm

As a result, the hybrid hollow capsule, 3L-HHC, according to the present invention showed a controlled release behavior of 13.5% on average every cycle for a total 6 cycles until all drugs were released. On the other hand, it was found that the hollow capsules for comparison, 3L-HC, showed 49.7% drug release at the first pressing cycle, and entire drug release at the third cycle of pressing.

### Example 9

### Drug Loading to Hollow Microcapsules and Drug Release by External Forces (Comparison of Hybrid Capsule (Chi-Alg-Chi)-(SiO2-Chi)3 with Control Capsule (Chi-Alg-Chi)-(Alg-Chi)3)

Fluorescein and fluorescently labeled fluorescein isothiocyanate (FITC) labeled dextran (MW: 4 kDa) could be used as a model drug having a low molecular weight and a model drug having a high molecular weight to be loaded in hollow microcapsules, respectively. The hollow capsules were dispersed in a 0.1 M NaCl solution in which the model drugs were dissolved in a concentration of 0.1 w/v % and left at room temperature for 12 hours, thereby loading the hollow capsules with the model drugs. Thereamong, as one example, drug release of the hollow microcapsules loaded with fluorescent labeled dextran was examined under external pressure.

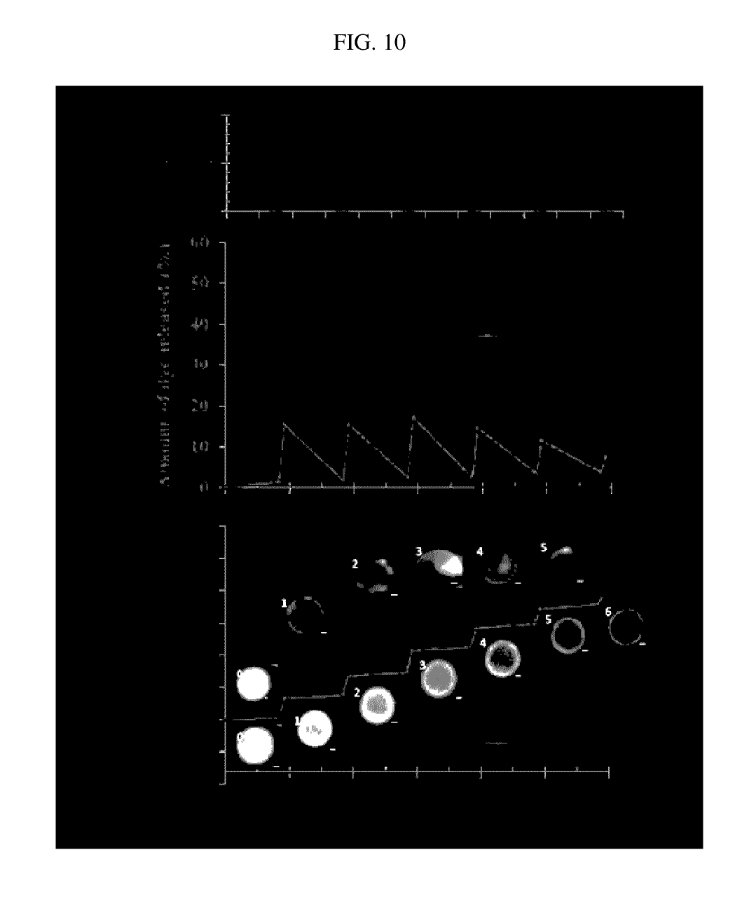

A glass surface was treated such that capsules could be attached thereto, and the drug-loaded capsules were evenly spread on the glass surface, followed by repeating application of a compressive pressure of 0.98 N for 6 seconds and relaxation without external force for 10 minutes in order to observe drug release (FIG. 10). In this experiment, two sorts of hollow microcapsules were used. That is, capsules comprising chitosan and alginate (Chi-Alg-Chi)-(Alg-Chi)₃ coatings were used as a control group, and hybrid hollow capsules (3L-HHC) including silica particles (Chi-Alg-Chi)-(SiO₂-Chi) 3 were used as an experimental group. Fluorescently labeled dextran released by each cycle of external force was quantified at a wavelength of 493 nm depending upon time (FIG. 10). The third graph in FIG. 10 is an accumulation graph obtained from results of the second graph in FIG. 10, wherein fluorescent microscope images of representative capsules at corresponding time points of each external cycle are shown.

Although some embodiments have been described herein, it should be understood by those skilled in the art that these embodiments are given by way of illustration only, and that various modifications, variations, and alterations can be made without departing from the spirit and scope of the invention. Therefore, the scope of the invention should be limited only by the accompanying claims and equivalents thereof.

1. A hollow microcapsule, comprising:
(a) a hollow core polymer layer, and
(b) an organic-inorganic complex layer comprising inorganic nanoparticles and a polymer for coating capsules on the surface of the hollow core polymer layer,
wherein the organic-inorganic complex layer is a single organic-inorganic complex layer or a plurality of organic-inorganic complex layers formed in a layer-by-layer manner, and
the core polymer layer and the polymer for coating capsules are crosslinked.

2. The hollow microcapsule according to claim 1, wherein the organic-inorganic complex layer comprises one or a plurality of organic-inorganic complex layers formed by alternately stacking (b1) an inorganic nanoparticle layer comprising inorganic nanoparticles and (b2) a polymer layer for coating capsules comprising the polymer for coating capsules at least once on the surface of the hollow core polymer layer.

3. The hollow microcapsule according to claim 2, wherein the hollow core polymer layer is (i) a single polymer core layer of a positively charged polymer, or (ii) a complex polymer core layer formed by alternately stacking a positively charged polymer layer and a negatively charged polymer layer at least once, and an outermost polymer layer of the complex polymer core layer is a positively charged polymer layer.

4. The hollow microcapsule according to claim 2, wherein the hollow core polymer layer is (i) a chitosan polymer core layer, or (ii) a complex polymer core layer formed by alternately stacking an alginic acid layer and a chitosan layer at least once on the hollow chitosan layer, and an outermost polymer layer of the complex polymer core layer is a chitosan polymer layer.

5. The hollow microcapsule according to claim 2, wherein the (b) organic-inorganic complex layer comprises 1 to 30 organic-inorganic complex layers of inorganic nanoparticle layers and polymer layers for coating capsules).

6. The hollow microcapsule according to claim 2, wherein the (b) organic-inorganic complex layer is selected from a complex layer formed by sequentially stacking 1 to 10 layers of silica layers and chitosan layers, a complex layer formed by sequentially stacking 1 to 10 layers of hydroxyapatite layers and chitosan layers, and a complex layer formed by sequentially stacking 1 to 10 layers of magnetite layers and chitosan layers.

7. The hollow microcapsule according to claim 2, further comprising: an outermost polymer layer on a surface of the outermost polymer layer for coating capsules.

8. The hollow microcapsule according to claim 1, wherein the organic-inorganic complex layer comprises one or a plurality of organic-inorganic complex layers formed by alternately stacking (b1′) an inorganic nanoparticle layer comprising the inorganic nanoparticles coated with a polymer for coating inorganic nanoparticles and (b2) the polymer layer for coating capsules once or repeatedly on the surface of the hollow core polymer layer, and
the polymer for coating inorganic nanoparticles is crosslinked.

9. The hollow microcapsule according to claim 8, wherein the hollow core polymer layer may be (i) a single polymer core layer of a negatively charged polymer, or (ii) a complex polymer core layer formed by alternately stacking a positively charged polymer layer and a negatively charged polymer layer at least once, and an outermost polymer layer of the complex polymer core layer is a negatively charged polymer layer.

10. The hollow microcapsule according to claim 8, wherein the hollow core polymer layer is an alginate single layer.

11. The hollow microcapsule according to claim 8, wherein (b) the organic-inorganic complex layer comprises 1 to 30 organic-inorganic complex layers of the coated inorganic nanoparticle layers and the polymer layers for coating capsules).

12. The hollow microcapsule according to claim 8, wherein the polymer for coating inorganic nanoparticles is a positively charged polymer and the polymer for coating capsules is a negatively charged polymer.

13. The hollow microcapsule according to claim 8, wherein the (b) organic-inorganic complex layer is a complex layer formed by sequentially stacking 1 to 10 layer of chitosan coated silica layers and alginate layers.

14. The hollow microcapsule according to claim 8, further comprising an outermost polymer layer on a surface of an outermost polymer layer for coating capsules, and the outermost polymer layer is a positively charged polymer layer.

15. The hollow microcapsule according to claim 1, wherein the hollow microcapsule has elasticity to be deformed in application of external force thereto and to be recovered to an original shape thereof when the external force is removed therefrom.

16. A drug delivery carrier comprising the hollow microcapsule according to claim 1.

17. The drug delivery carrier according to claim 16, wherein the drug delivery carrier responds to mechanical stimuli or is controllable by mechanical stimuli.

18. A method of preparing a hollow microcapsule, comprising:
(A) forming a core polymer layer on \textcircled{1} a positively charged sacrificial core or \textcircled{2} a negative charge-modified sacrificial core;
(B) \textcircled{1} if the sacrificial core is the positively charged sacrificial core, alternately forming an inorganic nanoparticle layer and a polymer layer for coating capsules at least once on the core polymer layer, and \textcircled{2} if the sacrificial core is the negative charge-modified sacrificial core, alternately forming an inorganic nanoparticle layer coated with a composition for coating inorganic nanoparticles and a polymer layer for coating capsules at least once on the core polymer layer;
(C) crosslinking the core polymer and the polymer for coating capsules; and
(D) removing the sacrificial core by etching.

19. The method of preparing a hollow microcapsule according to claim 18, wherein the (C) step is performed at subzero temperature.

20. The method of preparing a hollow microcapsule according to claim 18, wherein the positively charged sacrificial core is a calcium carbonate micro-particle, the negative charge-modified sacrificial core is a calcium carbonate micro-particle modified with phosphate, and the core polymer layer and the polymer layer for coating capsules are formed by a layer-by-layer method.
